(19) United States
(12) Patent Application Publication
(10) Pub. No.: US 2007/0292365 A1
(43) Pub. Date: Dec. 20, 2007

(54) METHOD OF MITIGATING AND DISRUPTING TARTAR BUILDUP

(76) Inventors: Tim Clarot, Phoenix, AZ (US); Regina Miskewitz, Phoenix, AZ (US)

Correspondence Address: SNELL & WILMER L.L.P. (Main), 400 EAST VAN BUREN, ONE ARIZONA CENTER, PHOENIX, AZ, 85004-2202, US

(21) Appl. No.: 11/749,103

(22) Filed: May 15, 2007

Related U.S. Application Data
(60) Provisional application No. 60/800,918, filed on May 15, 2006.

(51) Int. Cl. A61K 8/00; A61K 8/22; A61K 8/37; A61K 8/46; A61K 8/55; A61K 8/69; A61K 8/97; A61Q 11/00

(52) U.S. Cl. 424/52; 424/49; 424/53; 424/55; 424/56; 424/57; 424/58

(57) ABSTRACT

The present invention provides a method to reduce an amount of tartar on a surface and an amount of tartar buildup. The method includes applying a composition configured to maintain one or more active ingredients in contact with a portion of an oral cavity for an extended period of time. The composition may be applied using an applicator to selectively apply the composition to targeted areas within an oral cavity.

### CROSS REFERENCE TO RELATED APPLICATIONS

This application claims benefit to U.S. Provisional Application No. 60/800,918, entitled “COMPOSITION TO PREVENT AND REDUCE TARTAR BUILDUP AND METHOD OF USING SAME,” which was filed on May 15, 2006.

### FIELD OF INVENTION

The present invention generally relates to methods of using oral compositions for preventing tartar buildup and for removing existing tartar on surfaces such as teeth and dental devices. More particularly, the invention relates to methods of using viscous compositions that are designed to keep tartar removal or prevention agents proximate areas of the surface for an extended period of time.

### BACKGROUND OF THE INVENTION

Plaque can be defined as an organized, coherent, gel-like or mucoid material that includes microorganisms in an organic matrix derived from saliva and extracellular bacterial products such as glucans, fructans, enzymes, toxins, and acids. Plaque may also contain other cells, such as desquamated epithelial cells, and inorganic components, such as calcium and phosphate. In general, dental plaque is a bacterial accumulation. Generally transparent and sticky, plaque accumulates around the teeth at the cervical margin, and then grows apically.

Once plaque forms on a surface, the plaque resists removal, and usually can be removed only by mechanical means, such as, for example, by brushing and flossing the affected areas. If not removed, however, the presence of plaque can give rise to tartar formation, tooth decay, gingivitis, periodontitis, and other health problems.

Tartar, also referred to as calculus, is a hard, calcified plaque material that exhibits a yellowish or brownish color. Tartar forms as a result of minerals (e.g., those present in saliva and gum pockets) reacting with plaque material to form a rough calculus. Calculus generally arises from the nucleation of calcium phosphate, often in areas where the large salivary gland ducts secrete their saliva. As such, calculus can form on surfaces not covered by the oral mucosa (supragingival) or on surfaces located apical to the soft tissue margin of the gingiva (subgingival).

Tartar adheres to hard surfaces such as enamel, roots, and dental devices, such as dentures, bridges, crowns, and the like, and is generally more difficult to remove than plaque. Brushing and flossing are normally not sufficient to remove tartar from a surface.

If left untreated, tartar buildup can be problematic in several regards. For example, the rough, porous surface of tartar serves as a breeding ground for additional bacteria, which can calcify and form additional tartar. The bacteria growth can, in turn, lead to tooth decay, gum disease, tooth loss, as well as systemic health problems.

In addition to the health concerns, tartar can be a cosmetic problem due to its yellowish or brownish color, which appear as stains. Moreover, because the surface of tartar is rough and porous, the tartar absorbs colors from other sources (e.g., coffee, tea, smoke, tobacco, red wine and the like), and thus the presence of tartar exacerbates cosmetic tooth coloration typically associated with such other sources.

Typical methods of preventing tartar buildup include brushing with a tartar control toothpaste. Although such toothpastes, if used regularly, may prevent additional buildup of tartar, the toothpastes are not thought to be effective at removing existing tartar from tooth and device surfaces.

Methods of removing existing tartar typically include scaling or root planing, both of which are performed by dentists or hygienists with the aid of specialized tools. Although these techniques work well, they are relatively expensive and time consuming. Furthermore, various methods for inhibiting tartar crystals, may cause damage to tooth enamel and/or to dental devices.

Accordingly, improved techniques and compositions for preventing tartar buildup and removing tartar from surfaces are desired.

### SUMMARY OF THE INVENTION

The present invention provides a method for preventing and/or mitigating tartar buildup and for removing or disrupting existing tartar. More particularly, the invention provides a technique for using a composition that includes one or more active ingredients, for tartar removal or prevention of tartar buildup, in a viscous gel vehicle.

While the ways in which the invention addresses the various drawbacks of known compositions and methods will be described in more detail below, in general, the present invention provides a relatively inexpensive, safe composition for the prevention and reduction of tartar buildup. Compositions of the present invention and methods of using the compositions are relatively easy to use, may be applied at home, and do not damage the surface of enamel when used correctly.

In accordance with one embodiment of the invention, a method includes applying a composition including one or more active ingredients to prevent tartar build-up and/or reduce an amount of tartar on a surface and a carrier designed to maintain the active ingredient(s) in contact with the surface for an extended period of time. Exemplary compositions have a viscosity greater than about 20,000 cp, preferably greater than 30,000 cp, and more preferably greater than about 35,000 cp. The viscosity of the compositions may range from about 20,000 to about 250,000 cp, preferably about 25,000 to about 100,000, and more preferably about 30,000 to about 50,000 cp. Suitable active ingredients include cetylpyridinium chloride (CPC), zinc salts, other antimicrobial agents such as those described herein, and other ingredients known to reduce tartar and/or to reduce the formation of plaque.

In accordance with another embodiment of the invention, a method includes applying a composition including a plurality of ingredients to prevent or remove tartar and a carrier that includes a thickening agent. The composition is configured to maintain the plurality of active ingredients in contact with a surface for an extended period of time. Exemplary active ingredients include CPC, zinc salts, other antimicrobial agents, and similar agents known to reduce tartar or plaque. Exemplary thickeners include, for example, hydroxyethylcellulose, hydroxypropyl methylcellulose, carrageenan, sugar, guar gum, methylcellulose, methyethylecellulose, acceptable non-ionic thickeners, and/or the like.

In accordance with yet another embodiment of the present invention, a method of preventing or mitigating tartar buildup on a surface and for removing existing tartar includes applying a composition including a colorant indicative of healthy gingiva, an active ingredient for reducing and/or preventing tartar buildup, and a carrier. In accordance with various aspects of this embodiment, the viscosity of the composition is greater than about 20,000, preferably greater than 30,000 cp, and more preferably greater than about 35,000 cp. The viscosity of the compositions may range from about 20,000 to about 250,000 cp, preferably about 25,000 to about 100,000, and more preferably about 30,000 to about 50,000 cp.

In accordance with an additional embodiment of the invention, a method of preventing and/or mitigating tartar buildup on a surface includes applying a composition having a viscosity greater than about 20,000 cp directly to the surface. In accordance with various aspects of this embodiment, the composition is dispensed from a sealed container by rupturing the sealed container and applying a single dose of the composition directly to the surface. In accordance with further aspects of this embodiment, the active ingredient(s) include zinc salts, CPC, or other tartar removing and/or antimicrobial agents. In accordance with yet further aspects, the composition has a color indicative of healthy gums.

In accordance with an additional embodiment of the invention, a method of removing existing tartar buildup from a surface includes applying a composition having a viscosity greater than about 20,000 cp directly to the surface. In accordance with various aspects of this embodiment, the composition is dispensed from a sealed container by rupturing the sealed container and applying a single dose of the composition directly to the surface. In accordance with further aspects of this embodiment, the active ingredient(s) include zinc salts, CPC, or other tartar removing and/or antimicrobial agents. In accordance with yet further aspects, the composition has a color indicative of healthy gums.

BRIEF DESCRIPTION OF THE DRAWINGS

The exemplary embodiments of the present invention will be described in connection with the appended drawing figures in which like numerals denote like elements and:

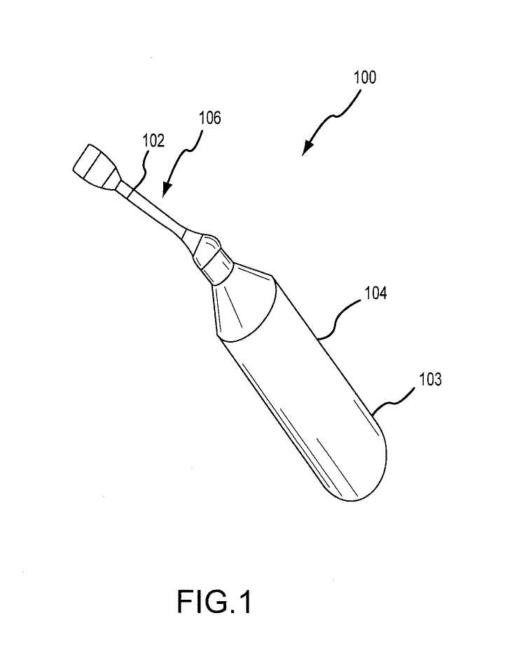

FIG. 1 illustrates a perspective view of a dispensing device for use in accordance with exemplary embodiments of the invention; and

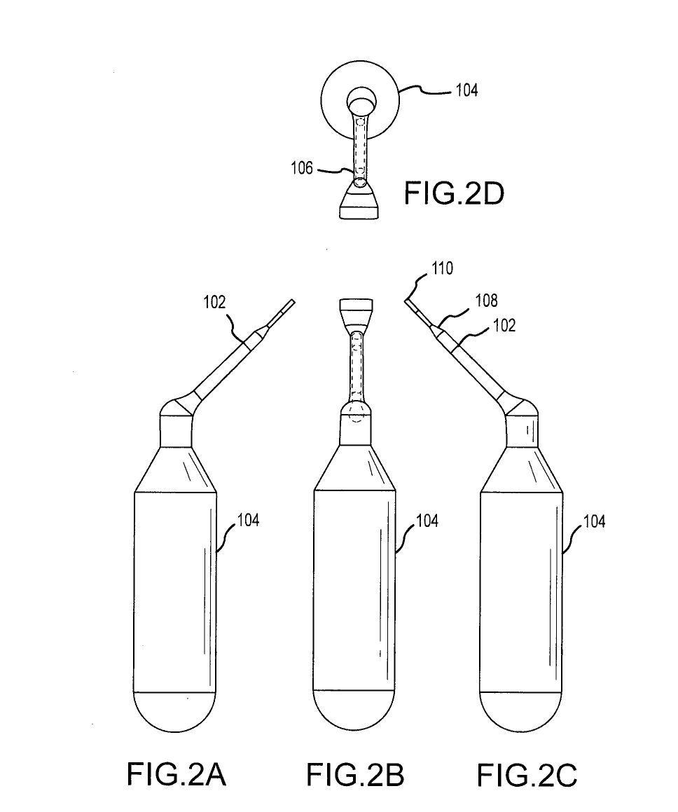

FIG. 2A-2D illustrate, respectively, left view, front view, right view and a top view of an exemplary container for use in accordance with an exemplary embodiment of the invention.

### DETAILED DESCRIPTION

The present invention provides a method of using an oral care composition that includes one or more active ingredients to prevent and/or reduce tartar buildup. As described herein, an exemplary method of the present invention is adapted to disrupt existing tartar accumulations, soften existing tartar, decrease the thickness of tartar accumulations, decrease a quality of tartar, remove existing tartar, and/or reduce or prevent tartar accumulation. The method of the invention can be used to reduce or prevent tartar buildup for various animals, and is particularly well suited for the treatment of humans.

As used throughout this application, the term surface includes any surface on which tartar may form. Exemplary surfaces include teeth (both supragingival and subgingival) and dental devices such as bridges, crowns, fillings, braces, and the like.

The present application refers to numerous aspects and/or embodiments of the invention, some of which are expressly described as being usable in combination with each other or under certain conditions. Any one or more of the features, aspects, and embodiments of the present disclosure may be used in combination with any of the other features, aspects, and/or embodiments. Moreover, references herein to the invention are to be understood broadly as examples of aspects of the present disclosure and not as limitations to the claims of the present disclosure.

Viscous Composition

In accordance with one embodiment of the invention, a composition for reducing, preventing, or mitigating tartar buildup includes an active ingredient effective at reducing, preventing, or mitigating tartar buildup and a viscous carrier. As explained in greater detail below, the composition is designed to maintain the active ingredient in contact with a surface for an extended period of time to allow the active ingredient(s) to stay in contact with possibly affected areas on the surface.

In accordance with various aspects of this embodiment, the active ingredients include one or more of the following: cetylpyridinium chloride (CPC), dicalcium phosphate dehydrate, hydrogen peroxide, sanguinaria extract, sodium bicarbonate, sodium lauryl sulfate, stannous fluoride, zinc salts such as zinc chloride, zinc acetate, zinc citrate, zinc oxide, and zinc gluconate, alkyl dimethyl amine oxide, alkyl dimethyl glycine, eucalyptol, menthol, methyl salicylate, thymol, sodium citrate, peppermint oil, sage oil, polymethylsiloxane, polxamer, and stannous pyrophosphate.

An amount of the active ingredient suitable for use with the composition varies in accordance with the dosage size and particular ingredient. In accordance with particular examples, an active ingredient is present in an amount of about 0.001 wt % to about 1.5 wt %, preferably about 0.025 wt % to about 1.0 wt %, and more preferably, about 0.05 wt % to about 0.7 wt %. All percentages set forth herein are in weight percent of the total composition, unless otherwise indicated.

In accordance with various aspects of this exemplary embodiment of the invention, the active ingredient(s) include CPC. In this case, the CPC is present in an amount of about 0.001% to about 1%, preferably about 0.01% to about 0.5%, and more preferably about 0.05% to about 0.25% or about 0.045% to about 0.1%. Additionally or alternatively, the active ingredient may include zinc gluconate. When included, the zinc gluconate is present in an amount of about 0.001% to about 1.5%, preferably about 0.01% to about 1.0%, and more preferably about 0.05% to about 0.75%.

In accordance with some implementations, the composition includes a thickener to obtain the desired viscosity. Suitable thickening agents include substances which increase the viscosity of the composition, cause the composition to gel or coagulate, or the like. Suitable thickeners include food-grade or pharmaceutical grade thickeners, including, for example, one or more of hydroxyethylcellulose, hydroxypropyl methylcellulose, carrageenan, guar gum, methylcellulose, methyethylecellulose, acceptable non-ionic thickeners, and the like. The thickener may be present in an amount of about 0.01 to about 10%, preferably about 0.1 to about 7%, and more preferably about 1% to about 5%.

The composition may also include a humectant, such as glycerin, which may be present in an amount of about 0.01% to about 15%, preferably about 0.1% to about 10%, and more preferably about 1 to about 7%. When used, the humectant may facilitate maintaining the composition in a liquid form and may help maintain a desired viscosity of the composition. In addition, glycerin may facilitate maintaining one or more of the active ingredients in an ionic form and may facilitate the transport of the active ingredients through the composition.

The composition may also include a suitable diluent. Exemplary diluents suitable for use with the present composition include water, alcohols, and oils. In accordance with particular examples of the invention, the composition includes purified water in an amount of about 80% to about 99%, preferably about 85% to about 95%, and more preferably about 88% to about 92%.

The composition may also include sugar alcohols, such as sorbitol, that act as a sweetener and also as a humectant and/or emulsifier. When used, sorbitol or other sugar alcohol is present in an amount of about 0.001% to about 5.0%, preferably about 0.01% to about 0. 1%, and more preferably about 0.025% to about 0.075%.

The composition may also include a natural or artificial sweetener such as sucralose, which, when included in the composition, is present in an amount of about 0.001% to about 1.5%, preferably about 0.01% to about 1%, and more preferably about 0.25% to about 0.75%. Other now known or hereafter devised sweeteners may also be utilized in effective amounts, such as, for example a natural or artificial sweetener such as saccharin, ace-k, aspartame, etc. The sweeteners, whether a natural or artificial sweetener, may also act as a humectant and/or emulsifier.

Colorants may also be added to the composition as desired. In accordance with various aspects, the composition includes colorants, such that when the composition is applied to or proximate the gingiva, the composition has a color indicative of healthy gingiva-i.e., the composition is pink in color. Providing a composition having a color indicative of healthy gingiva provides added incentive to users to continue using the composition, which in turn promotes removal and/or prevention of tartar buildup.

Colorants may be present in any desired amount. By way of particular example, the colorants may include Red #33 and/or Red #40, available from Pylam in an amount of about 0.00001% to about 0.05000%, preferably about 0.0007% to about 0.0010%, and more preferably about 0.00075% to about 0.00099%.

The composition also optionally includes additive or flavorants such as cinnamon oil, clove oil, mint flavorings, citrus flavorings, and the like. By way of one particular example, a composition may include cinnamon bark oil present in an amount of about 0.001 % to about 2%, preferably about 0.01% to about 1%, and more preferably about 0.1% to about 0.5%; and clove bud oil in an amount of about 0.001% to about 2%, preferably about 0.002% to about 1%, and more preferably about 0.003% to about 0.0075%. As used herein, the term flavorant refers also to sweeteners or other components that are added to affect the taste of the composition to the user. Similar to the description of the sweeteners above, flavorants of all types, including the oils and flavorings, may serve as a humectant and/or emulsifier in addition to modifying the taste of the composition.

The viscosity of the composition is configured to maintain the composition in contact with a surface for an extended period of time, which has several advantages over traditional compositions used to reduce or prevent tartar buildup. For example, because the composition is viscous and “sticks” to a surface to which it is applied, relatively small amounts of the composition and consequently the active agent(s) can be used to effectively prevent or remove tartar buildup on a surface. This is advantageous, not only from an economic viewpoint, but it also reduces an amount of the composition that may be ingested. Additionally, the viscosity of the composition enables easy delivery of the composition from its container or separate delivery device, while also facilitating maintaining the composition in contact with particular areas within an oral cavity, such as the crevices within teeth and between the teeth and at the gingival margin. In accordance with further embodiments of the invention, the viscosity is low enough to allow application of the composition to desired locations within the oral cavity.

Exemplary compositions have a viscosity greater than about 20,000 cp, preferably greater than 30,000 cp, and more preferably greater than about 35,000 cp. By way of more particular examples, the viscosity of compositions range from about 20,000 to about 250,000 cp, preferably about 25,000 to about 100,000, and more preferably about 30,000 to about 50,000 cp, and yet more preferably about 35,000 to about 45,000 cp. The viscosity values as set forth herein are measured at 25° C. using a Brookfield viscometer DV-II+ Pro, Spindle #6, 10 RPM, 90 seconds.

Within the viscosity ranges described above, the viscosity of the composition may be selected based on any one or more of a variety of factors, such as the cost of the ingredients, the safety of the ingredients and the risks of ingestion, the ease of application, and/or the preferences of the users. Additionally or alternatively, it may be determined that particular viscosities within the ranges described above work better with particular compositions within the scope of the present invention. For example, it may be determined that a less viscous composition is acceptable with certain active ingredients or certain concentrations of active ingredients while a more viscous composition is desired in others. For example, compositions within the scope of the present invention may have a preferred contact time of up to 10 hours, for example about 1 to about 10 hours or about 6 to about 8 hours per night for effective treatment. Compositions with a preferred contact time of just I hour may be less viscous than those that have a preferred contact time of 10 hours. The preferred contact time may vary depending on the active ingredient(s) and their concentration(s).

Additionally or alternatively, it may be determined that compositions including certain flavorants, whether sweeteners, oils, or other, are more effective at particular viscosities within the ranges described above. In some methods of using the present compositions, the compositions may be applied to the user's teeth or other dental surfaces at night and allowed to stay in contact with the surface while the user sleeps. Some flavorants or combinations of flavorants may cause the user to salivate more (or less) than a user would normally salivate at night. With such flavorants, it may be preferable to select a higher (or lower) composition viscosity to allow the composition to stay in contact with the surface for the desired amount of time even when the user is salivating more (or less) than normal. In some compositions within the scope of the present invention, the selection and quantity of flavorants and thickeners may be coordinated to reduce excess salivation by the user and/or to provide the desired viscosity and contact time in light of the expected user response. In some implementations, the compositions may be customized for individual users by adding additional thickeners and/or flavorants if the user has found that the originally provided composition produces unusually high salivation.

Multiple Active Ingredients

In accordance with another embodiment of the invention, the composition includes multiple active ingredients in a carrier, which includes a thickening agent. In this case, the composition includes a plurality of any of the active ingredients noted above and any thickening agent(s) noted above in the weight percents noted above. The thickener(s) may be added to produce a composition of any suitable viscosity. Exemplary viscosities and factors that contribute to the selection of a viscosity include those described above. The composition may also include any of the optional ingredients such as sweeteners, flavorants, and colorants as set forth herein.

By way of example, a composition in accordance with one embodiment of the invention includes CPC and zinc gluconate as the active ingredients. In this case, the CPC is present in an amount of about 0.001% to about 1%, preferably about 0.01% to about 0.5%, and more preferably about 0.05% to about 0.25% or 0.045% to about 0.1%; and the zinc gluconate is present in an amount of about 0.001% to about 1.5%, preferably about 0.01% to about 1.0%, and more preferably about 0.05% to about 0.75%.

Active Ingredient and Colorant

As noted above, compositions in accordance with various embodiments of the invention may include one or more active ingredients and a colorant indicative of healthy gingiva. When colorant is added, the color of the composition may be more than merely decorative. It serves the function of encouraging those that use the product to continue to use the product because there is an immediate appearance, upon application of the composition, that the composition is promoting healthy gingiva.

Method of Preventing Tartar Buildup

A method of preventing formation of tartar on a surface includes applying a composition including one or more of the active ingredients described herein to the surface and maintaining the composition in contact with the surface for an extended period of time.

The composition may be applied using a variety of methods such as with a swab, with a syringe, or using any other means capable of dispensing the composition. FIG. 1 and FIGS. 2A-2D illustrate a single dose dispensing device 100 suitable for applying the composition to a surface. The composition is dispensed from device 100 by severing or rupturing a portion of device 100 (e.g., at or near area 102 that has been scarred) to expose composition 104 contained within device 100. As noted above, a dose size may vary in accordance with several factors. Exemplary dose sizes for purposes of illustration range from about 2 mg to about 8 mg, preferably about 2 mg to about 6 mg, and more preferably about 2 mg to about 4 mg.

In accordance with various aspects of this embodiment, the composition is selectively applied though chute 106 to a surface, e.g., a tooth/gingiva interface, such that the composition contacts both supragingival and subgingival areas where tartar is likely to form. Unlike most methods of preventing tartar buildup, the method of this embodiment allows for direct, selective application of the composition to desired areas of a surface. Thus, less composition may be required to perform the task of preventing tartar buildup because the composition is not ubiquitously applied to an oral cavity.

Method of Reducing Tartar Buildup

A method of reducing an amount of tartar on a surface employs the same techniques described above, except that the composition is applied to areas on a surface affected by tartar.

### SPECIFIC EXAMPLE

The following non-limiting example illustrates an exemplary composition in accordance with various embodiments of the invention. This example is merely illustrative, and it is not intended that the invention be limited to the example. Compositions in accordance with the present invention may include the ingredients listed below as well as additional and/or alternative inert materials, preservatives, and other constituents typically found in compositions for removing and/or preventing tartar buildup. In the case where exemplary inert materials and/or preservatives are listed, these ingredients are merely exemplary, and it is understood that other similar ingredients may be substituted for the materials listed in the examples below.

### Example 1

### CPC and Zinc Gluconate

A pale light pink viscous gel, having a viscosity of about 40,000 cp, with cinnamon-clove characteristic odor and taste is formed by admixing the following ingredients in the amounts shown.

TABLE 1

| | | | Exemplary |
|---|---|---|---|
| Ingredient | Supplier | Weight % | Wt % Range |
| Purified Water | Copacker | 91.603 | 80-99 |
| Glycerin USP | Acme-Hardesty | 5.000 | 0.01-15 |
| HEC 250 HX | Hercules-Aqualon | 2.000 | 0.01-10 |
| Sorbitol | Roquette | 0.050 | 0.001-0.5 |
| Sucralose | Tate & Lyle | 0.400 | 0.001-1.5 |
| Cetylpyridinium | Dastech | 0.100 | 0.001-1 |
| Chloride | | | |
| Zinc Gluconate | American Ingredients/ | 0.592 | 0.001-1.5 |
| USP | Pharmachem Inc. | | |
| Cinnamon Bark | Berje | 0.250 | 0.001-2 |
| (Oil) | | | |
| Clove Bud (Oil) | Berje | 0.005 | 0.001-2 |
| Red #40 (1% sol.) | Pylam | 0.00099 | 0.00001-0.0500 |

A clinical study comparing the composition of Example 1 to a placebo and a mouth rinse including CPC was conducted. Each group started with 25 people each. Prior to a three-week evaluation, one person from the Example 1 group and one person from the mouth rinse group dropped out. At the conclusion of a three-week test period, there was an observable difference in the quality and thickness of calculus in the group using the composition of Example 1, compared to the placebo and the mouth rinse. A significant disruption of calculus bridges or masses of calculus was observed in subjects using the composition of Example 1. A softening of the calculus was also observed for those using the composition of Example 1.

In each instance, noticeable reductions in tartar and improvements were observed. In this regard, tartar reduction can be characterized as at least a twenty percent (20%) reduction on the Volpe-Manhold for calculus (V-MI) scale with use of the product over at least a six (6) week treatment period.

Although exemplary embodiments of the present invention are set forth herein, it should be appreciated that the invention is not so limited. Various modifications, variations, and enhancements in composition and method set forth herein may be made without departing from the spirit and scope of the present invention.

I claim:

1. A method of mitigating tartar accumulation on a surface, the method comprising:
providing a composition including a carrier, cetylpyridinium chloride, and a zinc salt;
applying the composition to a surface of a user; and
allowing the composition to remain in contact with the surface for an extended time.

2. The method of claim 1, wherein the composition has a viscosity greater than about 20,000 cp.

3. The method of claim 1, wherein the zinc salt is zinc gluconate.

4. The method of claim 3, wherein the zinc gluconate is present in an amount of about 0.001 wt % to about 1.5 wt % of the composition.

5. The method of claim 1, wherein the cetylpyridinium chloride is present in amount of about 0.001 wt % to about 1 wt % of the composition.

6. The method of claim 1, wherein the composition is applied to the surface with an auxiliary applicator.

7. The method of claim 1, wherein the composition is provided in a dosing applicator adapted to selectively dispense the composition to a surface within an oral cavity.

8. The method of claim 7, wherein a single dose amount is an amount between about 2 mg and about 8 mg of the composition.

9. A method of reducing tartar accumulation on a surface, the method comprising:
providing a composition having a viscosity between about 20,000 and about 250,000 cp and including a carrier and at least one active ingredient;
applying the composition to the surface; and
allowing the composition to remain in contact with the surface for an extended time.

10. The method of claim 9, wherein the at least one active ingredient is selected from the group consisting of cetylpyridinium chloride, dicalcium phosphate dehydrate, hydrogen peroxide, sanguinaria extract, sodium bicarbonate, sodium lauryl sulfate, stannous fluoride, zinc salts, alkyl dimethyl amine oxide, akyl dimethyl glycine, eucalyptol, menthol, methyl salicylate, thymol, sodium citrate, peppermint oil, sage oil, polymethylsiloxane, poloxamer, and pyrophosphates.

11. The composition of claim 9, wherein the at least one active ingredient is present in a combined amount of between about 0.001 wt % to about 2.5 wt %, of the composition.

12. The composition of claim 9, wherein the active ingredient includes zinc gluconate and cetylpyridinium chloride, wherein the zinc gluconate is present in an amount of about 0.001 wt % to about 1.5% of the composition, and wherein the cetylpyridinium chloride is present in amount of about 0.001 wt % to about 1 wt % of the composition.

13. The composition of claim 9, wherein the step of applying occurs prior to sleeping, and wherein the composition is allowed to remain in contact with the surface while the user is sleeping.

14. The method of claim 9, wherein the composition is provided in a dosing applicator adapted to selectively dispense single dose amounts of the composition and to direct dispensed amounts of the composition on targeted portions of the surface.

15. A method of disrupting tartar accumulation on a surface, the method comprising:
providing a viscous composition including a carrier, at least one active ingredient, at least one additive adapted to provide affirmative motivation to use the composition; and at least one thickener in a quantity sufficient to provide the composition with a viscosity between about 20,000 to about 250,000 cp;
applying a dose of the composition to a surface of a user; and
allowing the composition to remain in contact with the surface for an extended time.

16. The method of claim 15, wherein the at least one additive includes a colorant.

17. The method of claim 16, wherein the colorant provides the composition with a color indicative of healthy gingival.

18. The method of claim 16, wherein the colorant is present in an amount of about 0.00001 wt % to about 0.05000 wt %.

19. The method of claim 15, further comprising a flavorant.

20. The method of claim 19, wherein the flavorant is selected from the group consisting of cinnamon oil, clove oil, mint flavorings, citrus flavorings, sugar alcohols, natural sweeteners, and artificial sweeteners.
